(19) United States
(12) Patent Application Publication
(10) Pub. No.: US 2026/0232403 A1
(43) Pub. Date: Aug. 13, 2026

(54) TISSUE SENSING PROBE FOR FASTER, MORE ACCURATE REGISTRATIONS

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Craig A. Di Stefano, Andover, MA (US); Christopher J. Lynn, Austin, TX (US); Philip Cormier, Newburyport, MA (US); Nathan Zamarripa, Kittery Point, ME (US); Nicholas R. Labriola, Andover, MA (US); Geoffrey I. Karasic, Raynham, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/156,679

(22) PCT Filed: Apr. 18, 2024
(86) PCT No.: PCT/US2024/025193

§ 371 Date: Aug. 14, 2025

Related U.S. Application Data
(60) Provisional application No. 63/460,498, filed on Apr. 19, 2023.
(60) Provisional application No. 63/612,521, filed on Dec. 20, 2023.

(51) Int. Cl. A61B 90/00

(52) U.S. Cl. CPC A61B 90/06; A61B 2090/065; A61B 2090/3937; A61B 2562/0261

(57) ABSTRACT

A probe for registering an anatomical surface for a registration procedure includes a probe shaft, a probe tip having a base end coupled to a distal end of the probe shaft, a contact ball disposed and configured to rotate within a contact end of the probe tip, and a biasing element disposed within the probe tip. The biasing element is configured to bias the contact ball toward the contact end of the probe tip and the contact ball is configured to, in response to external force against the contact ball, push against the biasing element. The probe is configured to generate a signal in response to the contact ball pushing against the biasing element.

### CROSS-REFERENCE TO RELATED APPLICATIONS

This application claims the benefit of U.S. Provisional Application No. 63/460,498, filed on Apr. 19, 2023 and U.S. Provisional Application No. 63/612,521, filed on Dec. 20, 2023. The entire disclosures of the applications referenced above are incorporated herein by reference.

### FIELD

The present disclosure relates to probes for surgical systems, and more particularly to probes for registering points of an anatomical site for a surgical procedure.

### BACKGROUND

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Arthroscopic surgical procedures are minimally invasive surgical procedures in which access to the surgical site within the body is by way of small keyholes or ports through the patient's skin. The various tissues within the surgical site are visualized by way of an arthroscope placed through a port, and the internal scene is shown on an external display device. The tissue may be repaired or replaced through the same or additional ports. In computer-assisted surgical procedures (e.g., replacement of the anterior cruciate ligament (ACL), reduction of femora-acetabular impingement), the location of various objects with the surgical site may be tracked relative to the bone by way of images captured by an arthroscope and a three-dimensional model of the bone.

### SUMMARY

A probe for registering an anatomical surface for a registration procedure includes a probe shaft, a probe tip having a base end coupled to a distal end of the probe shaft, a contact ball disposed and configured to rotate within a contact end of the probe tip, and a biasing element disposed within the probe tip. The biasing element is configured to bias the contact ball toward the contact end of the probe tip and the contact ball is configured to, in response to external force against the contact ball, push against the biasing element. The probe is configured to generate a signal in response to the contact ball pushing against the biasing element.

In other features, a probe for registering an anatomical surface for a registration procedure includes a probe shaft, a probe tip having a contact end and a base end coupled to a distal end of the probe shaft, a plunger disposed within the probe tip that includes a flange at a proximal end of the plunger and a contact tip at a distal end of the plunger, and a biasing element disposed within the probe tip. The biasing element is configured to bias the plunger toward the contact end of the probe tip and the plunger is configured to, in response to external force against the contact tip, push against the biasing element. The probe is configured to generate a signal in response to the plunger pushing against the biasing element.

In other features, a probe for registering an anatomical surface for a registration procedure incudes a probe tip, a fiducial arranged on a body of the probe tip, an outer sleeve, at least a portion of body of the probe tip being enclosed and retained within the outer sleeve, an opening defined in the outer sleeve, and a biasing element arranged within the outer sleeve. The biasing element is configured to bias the probe tip toward a distal end of the probe such that the fiducial is detectable through the opening in response to pressure against the probe tip causing the fiducial to move to a detectable position within the opening.

In other features, a probe for registering an anatomical surface for a registration procedure includes a probe tip, an outer sleeve coupled to the probe tip, the outer sleeve defining an inner volume, an inner shaft retained within the outer sleeve, a biasing element arranged between a distal end of the inner shaft and a proximal end of the probe tip, the biasing element being configured to bias the inner shaft away from the probe tip, and a contact element arranged between a proximal end of the outer sleeve and a handle of the probe. The probe tip is configured to, in response to external force against the probe tip, push against the biasing element to cause the proximal end of the outer sleeve to contact the contact element.

In other features, a probe for registering an anatomical surface for a registration procedure includes a handle, a probe tip coupled to the handle via an inner shaft, a fiducial arranged on at least one of the probe tip and the inner shaft, and an outer sleeve. At least a portion of the probe tip and the inner shaft is enclosed within the outer sleeve, and the outer sleeve is slidable relative to the probe tip and the inner shaft such that the fiducial is exposed in a first position of the outer sleeve and the fiducial is not exposed in a second position of the outer sleeve relative.

In other features, a probe for registering an anatomical surface for a registration procedure includes an inner shaft, at least one fiducial arranged on the inner shaft, a probe tip coupled to the inner shaft, an outer sleeve enclosing the inner shaft, the outer sleeve including at least one opening, and a biasing element arranged within the outer sleeve between the inner shaft and the outer sleeve. The biasing element is configured to bias the inner shaft in first direction relative to the outer sleeve such that when external force is not applied to the probe tip, the inner shaft is biased into a first position in which the at least one fiducial is not detectable through the at least one opening, and when external force is applied to the probe tip, the biasing element is compressed and the inner shaft is biased into a second position in which the at least one fiducial is detectable through the at least one opening.

In other features, a probe for registering an anatomical surface for a registration procedure includes an inner shaft, at least one fiducial arranged on the inner shaft, a probe tip coupled to the inner shaft, an outer sleeve enclosing the inner shaft, the outer sleeve including at least one opening, a blocking assembly arranged within the outer sleeve, and a biasing element arranged within the outer sleeve between a distal end of the outer sleeve and a distal end of blocking assembly. When the biasing element is compressed, the blocking assembly is in a first position in which at least a portion of the at least one fiducial is covered by the blocking assembly and not detectable through the at least one opening, and the biasing element is configured to bias the blocking assembly into a second position in which the at least one fiducial is detectable through the at least one opening. A button is arranged to maintain the blocking assembly in the first position and selectively enable the blocking assembly to move into the second position.

In other features of the present disclosure, various methods are used to implement and perform functions related to the probes described herein. In other features, one or more systems, computing devices, processors or processing devices, etc. are configured to perform functions related to the probes described herein.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 shows a surgical system in accordance with at least some embodiments;

FIG. 2 shows a conceptual drawing of a surgical site with various objects within the surgical site tracked, in accordance with at least some embodiments;

FIG. 3 shows a method in accordance with at least some embodiments;

FIG. 4 is an example video display showing portions of a femur and a bone fiducial during a registration procedure, in accordance with at least some embodiments;

FIG. 5 shows a method in accordance with at least some embodiments;

FIGS. 6A, 6B, and 6C show example probe tips that include a roller ball tip or contact ball in accordance with at least some embodiments;

FIGS. 7A, 7B, and 7C show example probe tips that include a roller ball tip or contact ball in accordance with at least some embodiments;

FIG. 8 shows another example probe tip in accordance with at least some embodiments;

FIG. 9 shows another example probe assembly including a probe tip in accordance with at least some embodiments;

FIGS. 10A, 10B, 10C, and 10D show another example probe assembly in accordance with at least some embodiments;

FIGS. 11A and 11B show another example probe assembly in accordance with at least some embodiments;

FIG. 12 shows a computer system in accordance with at least some embodiments; and

FIG. 13 illustrates steps of an example method for performing a touch-based registration procedure in accordance with at least some embodiments.

In the drawings, reference numbers may be reused to identify similar and/or identical elements.

### DEFINITIONS

Various terms are used to refer to particular system components. Different companies may refer to a component by different names-this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

An endoscope having “a single optical path” through an endoscope shall mean that the endoscope is not a stereoscopic endoscope having two distinct optical paths separated by an interocular distance at the light collecting end of the endoscope. The fact that an endoscope has two or more optical members (e.g., glass rods, optical fibers) forming a single optical path shall not obviate the status as a single optical path.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various examples are directed to methods and systems of registering a three-dimensional model of a rigid structure, such as bone. More particularly, various examples are directed to methods and related systems of identifying surface features of a rigid structure visible in a video stream, and using the surface features to register a three-dimensional model for use in computer-assisted navigation of the surgical procedure. In some examples, the surface features are determined using touchless techniques based on a known or calculated motion of the camera. In other examples, the surface features are gathered using a touch probe that is not itself directly tracked; but rather, the pose of the touch probe, and thus the locations of the distal tip of the touch probe touching the bone, may be determined by segmenting the frames of the video stream and pose estimation. In yet still further examples, the three-dimensional model may be registered by use of a patient-specific instrument that couples to the rigid structure in only one orientation; thus, a fiducial coupled to the patient-specific instrument, or in some cases the patient-specific instrument itself without a fiducial, may be used to register the three-dimensional bone model.

The various examples were developed in the context of anterior-cruciate ligament (ACL) repair (e.g., for placing femoral and/or tibial tunnels during ACL reconstruction), and thus the discussion below is based on the developmental context. In this context, the rigid structure is bone, and the three-dimensional mode is a three-dimensional bone model. However, the techniques are applicable to any suitable rigid anatomical structure, such as teeth. Moreover, the various techniques may be applicable to many types of surgical procedures, such as repairs associated with the knee, the hip, the shoulder, the wrist, or the ankle. The techniques may be applicable not only to ligament repair (e.g., medial collateral ligament repair, lateral collateral ligament repair, and posterior cruciate ligament repair), but also for planning and placing anchors to reattach soft tissue (e.g., reattaching the labrum of the hip, the rotator cuff, or the meniscal root), and surgical procedures to address femoroacetabular impingement. Thus, the description and developmental context shall not be read as a limitation of the applicability of the teachings. In order to orient the reader, the specification first turns a description of the knee.

In some examples of the registration procedure, a user probes an anatomical surface using a handheld probe. Collected points (e.g., a point cloud) are processed (e.g., using a machine learning algorithm) and matched to a bone model, such as a bone model created via a scan (e.g., a CT or MRI scan) or other technique. For example, the bone model is overlaid on top of a live arthroscopic video feed to provide an augmented or mixed reality visual representation of a surgical or anatomical site. During conventional implementations of the registration procedure, the user collects points by manual inputting commands to the surgical system to start, pause, and end collection using a tablet or other computing device (e.g., as controlled by nursing or other surgical staff), arthroscope camera head buttons, etc. Accordingly, the registration procedure reduces the speed and efficiency of the overall surgical procedure.

In some examples, systems and methods according to the present disclosure implement a connected or “smart” probe and probe tip (referred to collectively as a “probe assembly”) configured to facilitate the registration procedure. When the probe tip is pressed against tissue, the surgical system can detect, automatically, whether the probe is in contact with the surface (e.g., bone) and provides feedback to the system, in real-time (e.g., via coded or wireless communication, wired communication, etc.). In an example, the probe tip is configured for atraumatic contact with anatomical surfaces. In other words, the probe tip does not include pointed or edged geometry (e.g., a needle or blade) configured to pierce or cut tissue. Rather, the probe tip is blunted or rounded to facilitate contact with the bone or other surface without damaging adjacent tissue. In some examples, the probe and/or probe tip is disposable.

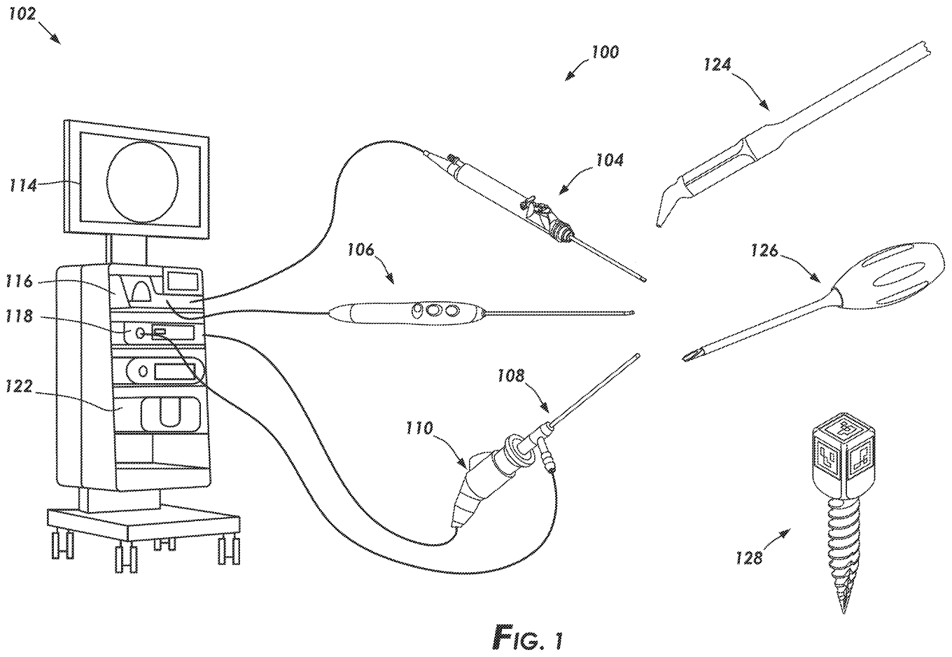

FIG. 1 shows a surgical system (not to scale) 100 in accordance with at least some embodiments. In particular, the example surgical system 100 comprises a tower or device cart 102, an example mechanical resection instrument 104, an example plasma-based ablation instrument (hereafter just ablation instrument 106), and an endoscope in the example form of an arthroscope 108 and attached camera head or camera 110. In the example systems, the arthroscope 108 is a rigid device, unlike endoscopes for other procedures, such as upper-endoscopies. The device cart 102 may comprise a display device 414, a resection controller 116, and a camera control unit (CCU) together with an endoscopic light source and video controller 118. In example cases the combined CCU and video controller 118 not only provides light to the arthroscope 108 and displays images received from the camera 110, but also implements various additional aspects, such as registering a three-dimensional bone model with the bone visible in the video images, and providing computer-assisted navigation during the surgery. Thus, the combined CCU and video controller are hereafter referred to as surgical controller 118. In other cases, however, the CCU and video controller may be a separate and distinct system from the controller that handles registration and computer-assisted navigation, yet the separate devices would nevertheless be operationally coupled.

The example device cart 102 further includes a pump controller 122 (e.g., single or dual peristaltic pump). Fluidic connections of the mechanical resection instrument 104 and ablation instrument 106 to the pump controller 122 are not shown so as not to unduly complicate the figure. Similarly, fluidic connections between the pump controller 122 and the patient are not shown so as not to unduly complicate the figure. In the example system, both the mechanical resection instrument 104 and the ablation instrument 106 are coupled to the resection controller 116 being a dual-function controller. In other cases, however, there may be a mechanical resection controller separate and distinct from an ablation controller. The example devices and controllers associated with the device cart 102 are merely examples, and other examples include vacuum pumps, patient-positioning systems, robotic arms holding various instruments, ultrasonic cutting devices and related controllers, patient-positioning controllers, and robotic surgical systems.

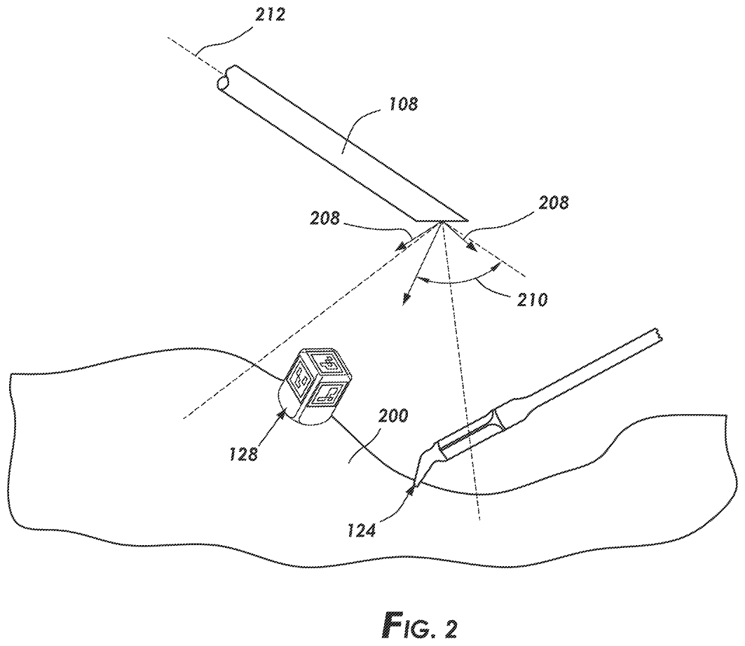

FIG. 2 further shows additional instruments that may be present during an arthroscopic surgical procedure. In particular, FIG. 2 shows an example touch probe 124, a drill guide or aimer 126, and a bone fiducial 128. The touch probe 124 may be used during the surgical procedure to provide information to the surgical controller 118, such as information to register a three-dimensional bone model to an underlying bone visible in images captured by the arthroscope 108 and camera head 110. The aimer 126 may be used as a guide for placement and drilling with a drill wire to create an initial or pilot tunnel through the bone. The bone fiducial 128 may be affixed or rigidly attached to the bone and serve as an anchor location for the surgical controller 118 to know the orientation of the bone (e.g., after registration of a three-dimensional bone model). Additional tools and instruments will be present, such as the drill wire, various reamers for creating the throughbore and counterbore aspects of a tunnel through the bone, and various tools, such as for suturing and anchoring a graft. These additional tools and instruments are not shown so as not to further complicate the figure. The specification now turns to a workflow for an example anterior cruciate ligament repair.

A surgical procedure may begin with a planning phase. The example anterior cruciate ligament repair may start with imaging (e.g., X-ray imaging, computed tomography (CT), magnetic resonance imaging (MRI)) of the knee of the patient, including the relevant anatomy like the lower portion of the femur, the upper portion of the tibia, and the articular cartilage. The imaging may be preoperative imaging, hours or days before the intraoperative repair, or the imaging may take place within the surgical setting just prior to the intraoperative repair. The discussion that follows assumes MRI imaging, but again many different types of imaging may be used. The image slices from the MRI imaging can be segmented such that a volumetric model or three-dimensional model of the anatomy is created. Any suitable currently available, or after developed, segmentation technology may be used to create the three-dimensional model. More specifically to the example of anterior cruciate ligament repair, a three-dimensional bone model of the lower portion of the femur, including the femoral condyles, is created.

Using the three-dimensional bone model, an operative plan is created that comprises choosing a planned-tunnel path through the femur, including locations of the apertures of the bone that define the ends of the tunnel. For an example inside-out repair, the aperture within the femoral notch is the entry location for the drilling, and the aperture on the lateral surface of the femur is the exit location. For an outside-in repair, the entry and exit locations for drilling are swapped. Still assuming an inside-out repair, the entry location may be selected to be the same as, or close to, the attachment location of the native anterior cruciate ligament to the femur within the femoral notch. In some cases, selecting the entry location within the femoral notch may involve use of a Bernard & Hertel Quadrant or grid placed on a fluoroscopic image, or by placing the Bernard & Hertel Quadrant on a simulated fluoroscopic image created from the three-dimensional bone model. Based on use of the Bernard & Hertel Quadrant, an entry location for the tunnel is selected. For an inside-out repair, selection of the exit location is less restrictive, not only because the portion of the tunnel proximate to the exit location is used for placement of the anchor for the graft, but also because the exit location is in approximately centered in the femur (considered anteriorly to posteriorly), and thus issues of bone wall thickness at the exit location are of less concern. In some cases, a three-dimensional bone model of the proximal end of the tibia is also created, and the surgeon may likewise choose planned-tunnel path(s) through the tibia.

The results of the planning may include: a three-dimensional bone model of the distal end of the femur; a three-dimensional bone model for a proximal end of the tibia; an entry location and exit location through the femur and thus a planned-tunnel path for the femur; and an entry location and exit location through the tibia and thus a planned-tunnel path through the tibia. Other surgical parameters may also be selected during the planning, such as tunnel throughbore diameters, tunnel counterbore diameters and depth, desired post-repair flexion, and the like, but those additional surgical parameters are omitted so as not to unduly complicate the specification.

The specification now turns to intraoperative aspects. The intraoperative aspects include steps and procedures for setting up the surgical system to perform the various repairs. It is noted, however, that some of the intraoperative aspects (e.g., optical system calibration), may take place before any ports or incisions are made through the patient's skin, and in fact before the patient is wheeled into the surgical room. Nevertheless, such steps and procedures may be considered intraoperative as they take place in the surgical setting and with the surgical equipment and instruments used to perform the actual repair.

The example ACL repair is conducted arthroscopically and is computer-assisted in the sense the surgical controller 118 is used for arthroscopic navigation within the surgical site. More particularly, in example systems the surgical controller 118 provides computer-assisted navigation during the ligament repair by tracking location of various objects within the surgical site, such as the location of the bone within the three-dimensional coordinate space of the view of the arthroscope, and location of the various instruments (e.g., a drill wire) within the three-dimensional coordinate space of the view of the arthroscope. The specification turns to brief description of such tracking techniques.

FIG. 2 shows a conceptual drawing of a surgical site with various objects within the surgical site. In particular, visible in FIG. 2 is a distal end of the arthroscope 108, a portion of a bone 200 (e.g., femur), the bone fiducial 128 within the surgical site, and the touch probe 124. Each is addressed in turn.

The arthroscope 108 illuminates the surgical site with visible light. In the example of FIG. 2, the illumination is illustrated by arrows 208. The illumination provided to the surgical site is reflected by various objects and tissues within the surgical site, and the reflected light that returns to the distal end enters the arthroscope 108, propagates along an optical channel within the arthroscope 108, and is eventually incident upon a capture array within the camera 110 (FIG. 1). The images detected by the capture array within the camera 110 are sent electronically to the surgical controller 118 (FIG. 1) and displayed on the display device 414 (FIG. 1). In one example, the arthroscope 108 is monocular or has a single optical path through the arthroscope for capturing images of the surgical site, notwithstanding that the single optical path may be constructed of two or more optical members (e.g., glass rods, optical fibers). That is to say, in example systems and methods the computer-assisted navigation provided by the arthroscope 108, the camera 110, and the surgical controller 118 is provided with the arthroscope 108 that is not a stereoscopic endoscope having two distinct optical paths separated by an interocular distance at the distal end endoscope.

During a surgical procedure, a surgeon selects an arthroscope with a viewing direction beneficial for the planned surgical procedure. Viewing direction refers to a line residing at the center of an angle subtended by the outside edges or peripheral edges of the view of an endoscope. The viewing direction for some arthroscopes is aligned with the longitudinal central axis of the arthroscope, and such arthroscopes are referred to as “zero degree” arthroscopes (e.g., the angle between the viewing direction and the longitudinal central axis of the arthroscope is zero degrees). The viewing direction of other arthroscopes forms a non-zero angle with the longitudinal central axis of the arthroscope. For example, for a 30o arthroscope the viewing direction forms a 30o angle to the longitudinal central axis of the arthroscope, the angle measured as an obtuse angle beyond the distal end of the arthroscope. In many cases for ACL repair, the surgeon selects a 30o arthroscope or a 45o arthroscope based on location the port created through the skin of the patient. In the example of FIG. 2, the view angle 210 of the arthroscope 108 forms a non-zero angle to the longitudinal central axis 212 of the arthroscope 108.

Still referring to FIG. 2, within the view of the arthroscope 108 is a portion of the bone 200 (e.g., within the intercondylar notch), along with the example bone fiducial 128, and the example touch probe 124. The example bone fiducial 128 is multi-faceted element, with each face or facet having a fiducial disposed or created thereon. However, the bone fiducial need not have multiple faces, and in fact may take any shape so long as that shape can be tracked within the video images. The bone fiducial, such as bone fiducial 128, may be attached to the bone 200 in any suitable form, in this example the fastening by the screw portion of the bone fiducial 128 (not visible in FIG. 2, but visible in FIG. 1). The patterns of the fiducials on each facet are designed to provide information regarding the orientation of the bone fiducial 128 in the three-dimensional coordinate space of the view of the arthroscope 108. More particularly, the pattern is selected such that the orientation of the bone fiducial 128 may be determined from images captured by the arthroscope 108 and attached camera (FIG. 1).

The touch probe 124 is also shown as partially visible within the view of the arthroscope 108. The touch probe 124 may be used, as discussed more below, to identify a plurality of surface features on the bone 200 as part of the registration of the bone 200 to the three-dimensional bone model. Alternatively, though not specifically shown, the aimer 126 (FIG. 1) may be used as the device to help with the registration process. In some cases the touch probe 124 and/or the aimer 126 may carry their own, unique fiducials, such that their respective poses may be calculated from the one or more fiducial present in the video stream. However, in other cases, and as shown, the medical instrument used to help with registration of the three-dimensional bone model, be it the touch probe 124, the aimer 126, or any other suitable medical device, may omit carrying fiducials. Stated otherwise, in such examples the medical instrument has no fiducial markings. In such cases, the pose of the medical instrument may be determined by a machine learning model, discussed in more detail below.

The images captured by the arthroscope 108 and attached camera are subject to optical distortion in many forms. For example, the visual field between distal end of the arthroscope 108 and the bone 200 within the surgical site is filled with fluid, such as bodily fluids and saline used to distend the joint. Many arthroscopes have one or more lenses at the distal end that widen the field of view, and the wider field of view causes a “fish eye” effect in the captured images. Further, the optical elements within the arthroscope (e.g., rod lenses) may have optical aberrations inherent to the manufacturing and/or assembly process. Further still, the camera may have various optical elements for focusing the images received onto the capture array, and the various optical elements may have aberrations inherent to the manufacturing and/or assembly process. In example systems, prior to use within each surgical procedure, the endoscopic optical system is calibrated to account for the various optical distortions. The calibration creates a characterization function that characterizes the optical distortion, and further analysis of the frames of the video stream may be, prior to further analysis, compensated using the characterization function.

The next example step in the intraoperative procedure is the registration of the bone model created during the planning stage. During the intraoperative repair, the three-dimensional bone model is obtained by or provided to the surgical controller 118. Again using the example of anterior cruciate ligament repair, and specifically computer-assisted navigation for tunnel paths through the femur, the three-dimensional bone model of the lower portion of the femur is obtained by or provided to the surgical controller 118. Thus, the surgical controller 118 receives the three-dimensional bone model, and assuming the arthroscope 108 is inserted into the knee by way of a port through the patient's skin, the surgical controller 118 also receives video images of a portion of the lower end of the femur. In order to relate the three-dimensional bone model to the images received by way of the arthroscope 108 and camera 110, the surgical controller 118 registers the three-dimensional bone model to the images of the femur received by way of the arthroscope 108 and camera 110.

In order to perform the registration, and in accordance with example methods, the bone fiducial 128 is attached to the femur. The bone fiducial placement is such that the bone fiducial is within the field of view of the arthroscope 108, but in a location spaced apart from the expected tunnel entry/exit point through the lateral condyle. More particularly, in example cases the bone fiducial 128 is placed within the intercondylar notch superior to the expected location of the tunnel through lateral condyle. To relate or register bone visible in the video images to the three-dimensional bone model, the surgical controller 118 (FIG. 1) is provided or determines a plurality of surface features of an outer surface of the bone. Identifying the surface features may take several forms, including a touch-based registration using the touch probe 124 without a carried fiducial, a touchless registration technique in which the surface features are identified after resolving the motion of the arthroscope 108 and camera relative to the bone fiducial 128, and a third technique in which uses a patient-specific instrument.

In the example touch-based registration, the surgeon may touch a plurality of locations using the touch probe 124 (FIG. 1). In some cases, particularly when portions of the outer surface of the bone are exposed to view, receiving the plurality of surface features of the outer surface of the bone may involve the surgeon “painting” the outer surface of the bone. “Painting” is a term of art that does not involve application of color or pigment, but instead implies motion of the touch probe 124 when the distal end of the touch probe 124 is touching bone. In this example, the touch probe 124 does not carry or have a fiducial visible to the arthroscope 108 and the camera 110. It follows that the pose of the touch probe 124 and the location of the distal tip of the touch probe 124 needs to be determined in order to gather the surface features for purposes of registering the three-dimensional bone model.

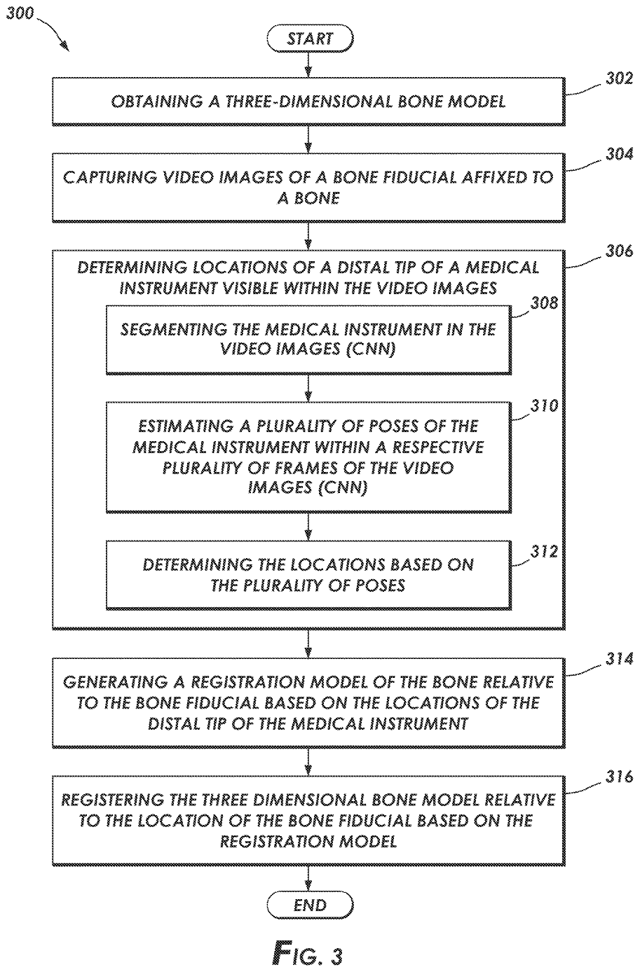

FIG. 3 shows a method 300 in accordance with at least some embodiments. The example method 300 may be implemented in software within a computer system, such as the surgical controller 118. In particular, the example method 300 comprises obtaining a three-dimensional bone model (block 302). That is to say, in the example method 300, what is obtained is the three-dimensional bone model that may be created by segmenting a plurality of non-invasive images (e.g., CT, MRI) taken preoperatively or intraoperatively. With the bone segmented from or within the images, the three-dimensional bone model may be created. The three-dimensional bone may take any suitable form, such as a computer-aided design (CAD) model, a point cloud of data points with respect to an arbitrary origin, or a parametric representation of a surface expressed using analytical mathematical equations. Thus, the three-dimensional bone model is defined with respect to the origin and in any suitable an orthogonal basis.

The next step in the example method 300 is capturing video images of the bone fiducial attached to the bone (block 304). The capturing is performed intraoperatively. In the example case of an arthroscopic anterior cruciate ligament repair, the capturing of video images is by way of the arthroscope 108 and camera 110. Other endoscopes may be used, such as endoscopes in which the capture array resides at the distal end of the device (e.g., chip-on-the-tip devices). However, in open procedures where the skin is cut and pulled away, exposing the bone to the open air, the capturing may be by any suitable camera device, such as one or both cameras of a stereoscopic camera system, or a portable computing device, such as a tablet or smart-phone device. The video images may be provided to the surgical controller 118 in any suitable form.

The next step in the example method 300 is determining locations of a distal tip of the medical instrument visible within the video images (block 306), where the distal tip is touching the bone in at least some of the frames of the video images, and the medical instrument does not have a fiducial. Determining the locations of the distal tip of the medical instrument may take any suitable form. In one example, determining the locations may include segmenting the medical instrument in the frames of the video images (block 308). The segmenting may take any suitable form, such as applying the video images to a segmentation machine learning algorithm. The segmentation machine learning algorithm may take any suitable form, such as neural network or convolution neural network trained with a training data set showing the medical instrument in a plurality of known orientations. The segmentation machine learning algorithm may produce segmented video images where the medical instrument is identified or highlighted in some way (e.g., box, brightness increased, other objects removed).

With the segmented video images, the example method 300 may estimate a plurality of poses of the medical instrument within a respective plurality of frames of the video images (block 310). The estimating the poses may take any suitable form, such as applying the video images to a pose machine learning algorithm. The pose machine learning algorithm may take any suitable form, such as neural network or convolution neural network trained to perform six-dimensional pose estimation. The resultant of the pose machine learning algorithm may be, for at least some of the frames of the video image, an estimated pose of the medical instrument in the reference frame of the video images and/or in the reference frame provided by the bone fiducial. That is, the resultant of the pose machine learning algorithm may be a plurality of poses, one pose each for at least some of the frames of the segmented video images. While in many cases a pose may be determined for each frame, in other cases it may not be possible to make a pose estimation for at least some frame because of video quality issues, such as motion blur caused by electronic shutter operation.

The next step in the example method 300 is determining the locations based on the plurality of poses (block 312). In particular, for each frame for which a pose can be estimated, based on a model of the medical device the location of the distal tip can be determined in the reference frame of the video images and/or the bone fiducial. Thus, the resultant is a set of locations that, at least some of which, represent locations of the outer surface of the bone.

The FIG. 3 shows an example three-step process for determining the locations of the distal tip of the medial instrument. However, the method 300 is merely an example, and many variations are possible. For example, a single machine learning model, such as a convolution neural network, may be set up and trained to perform all three steps as a single overall process, though there may be many hidden layers of the convolution neural network. That is, the convolution neural network may segment the medical instrument, perform the six-dimensional pose estimation, and determine the location of the distal tip in each frame. The training data set in such a situation would include a data set in which each frame has the medical device segmented, the six-dimensional pose identified, and the location of the distal tip identified. The output of the determining step 306 may be a segmented video stream distinct from the video images captured at step 304. In such cases, the later method steps may use both segmented video stream and the video images to perform the further tasks. In other cases, the location information may be combined with the video images, such as being embedded in the video images, or added as metadata to each frame of the video images.

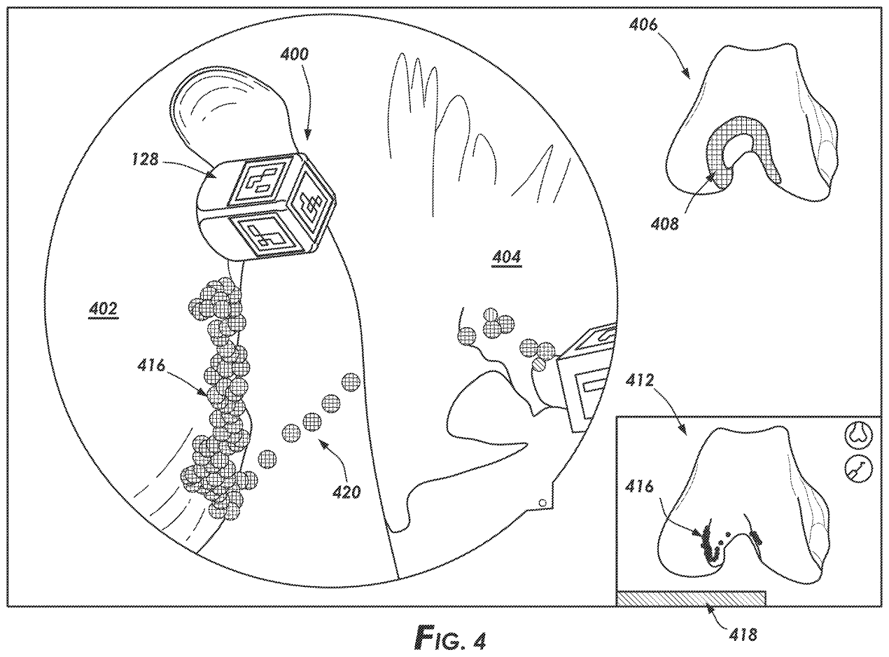

FIG. 4 is an example video display showing portions of a femur and a bone fiducial during a registration procedure. The display may be shown, for example, on the display device 114 associated with the device cart 102, or any other suitable location. In particular, visible in the main part of the display of FIG. 4 is an intercondylar notch 400, a portion of the lateral condyle 402, a portion the medial condyle 404, and the example bone fiducial 128. Shown in the upper right corner of the example display is a depiction of the bone, which may be a rendering 406 of the bone created from the three-dimensional bone model. Shown on the rendering 406 is a recommended area 408, the recommended area 408 being portions of the surface of the bone to be “painted” as part of the registration process. Shown in the lower right corner of the example display is a depiction of the bone, which again may be a rendering 412 of the bone created from the three-dimensional bone model. Shown on the rendering 412 are a plurality of surface features 416 on the bone model that have been identified as part of the registration process. Further shown in the lower right corner of the example display is progress indicator 418, showing the progress of providing and receiving of locations on the bone. The example progress indicator 418 is a horizontal bar having a length that is proportional to the number of locations received, but any suitable graphic or numerical display showing progress may be used (e.g., 0% to 100%).

Referring to both the main display and the lower right rendering, as the surgeon touches the outer surface of the bone within the images captured by the arthroscope 108 and camera 110, the surgical controller 118 receives the surface features on the bone, and may display each location both within the main display as dots or locations 416, and within the rendering shown in the lower right corner. More specifically, the example surgical controller 118 overlays indications of identified surface features 416 on the display of the images captured by the arthroscope 108 and camera 110, and in the example case shown, also overlays indications of identified surface features 416 on the rendering 412 of the bone model. Moreover, as the number of identified locations 416 increases, the surgical controller 118 also updates the progress indicator 418.

Still referring to FIG. 4, in spite of the diligence of the surgeon, not all locations identified by the surgical controller 118 based on the surgeon's movement of the touch probe 124 result in valid locations on the surface of the bone. In the example of FIG. 4, as the surgeon moves the touch probe 124 from the inside surface of the lateral condyle 102 to the inside surface of the medial condyle 104, the surgical controller 118, based on the example six-dimensional pose estimation, receives several locations 420 that likely represent locations at which the distal end of the touch probe 124 was not in contact with the bone.

Returning to FIG. 3, the plurality of surface features 416 may be, or the example surgical controller 118 may generate, a registration model relative to the bone fiducial 128 (block 314). The registration model may take any suitable form, such as a computer-aided design (CAD) model or point cloud of data points in any suitable orthogonal basis. The registration model, regardless of the form, may have fewer overall data points or less “structure” than the bone model created by the non-invasive computer imaging (e.g., MRI). However, the goal of the registration model is to provide the basis for the coordinate transforms and scaling used to correlate the bone model to the registration model and relative to the bone fiducial 128. Thus, the next step in the example method 300 is registering the bone model relative to the location of the bone fiducial based on the registration model (block 316). Registration may conceptually involve testing a plurality of coordinate transformations and scaling values to find a correlation that has a sufficiently high correlation or confidence factor. Once a correlation is found with the sufficiently high confidence factor, the bone model is said to be registered to the location of the bone fiducial. Thereafter, the example registration method 300 may end (block 318); however, the surgical controller 118 may then use the registered bone model to provide computer-assisted navigation regarding a procedure involving the bone.

In the examples discussed to this point, registration of the bone model involves a touch-based registration technique using the touch probe 124 without a carried fiducial. However, other registration techniques are possible, such as a touchless registration technique. The example touchless registration technique again relies on placement of the bone fiducial 128. As before, when the viewing direction of the arthroscope 108 is relatively constant, the bone fiducial may have fewer faces with respective fiducials. Once placed, the bone fiducial 128 represents a fixed location on the outer surface of the bone in the view of the arthroscope 108, even as the position of the arthroscope 108 is moved and changed relative to the bone fiducial 128. Again, in order to relate or register the bone visible in the video images to the three-dimensional bone model, the surgical controller 118 (FIG. 1) determines a plurality of surface features of an outer surface of the bone, and in this example determining the plurality of surface features is based on a touchless registration technique in which the surface features are identified based on motion of the arthroscope 108 and camera 110 relative to the bone fiducial 128.

Another technique for registering the bone model to the bone uses a patient-specific instrument. In both touch-based and touchless registration techniques, a registration model is created, and the registration model is used to register the bone model to the bone visible in the video images. Conceptually, the registration model is used to determine a coordinate transformation and scaling to align the bone model to the actual bone. However, if the orientation of the bone in the video images is known or can be determined, use of the registration model may be omitted, and instead the coordinate transformations and scaling may be calculated directly.

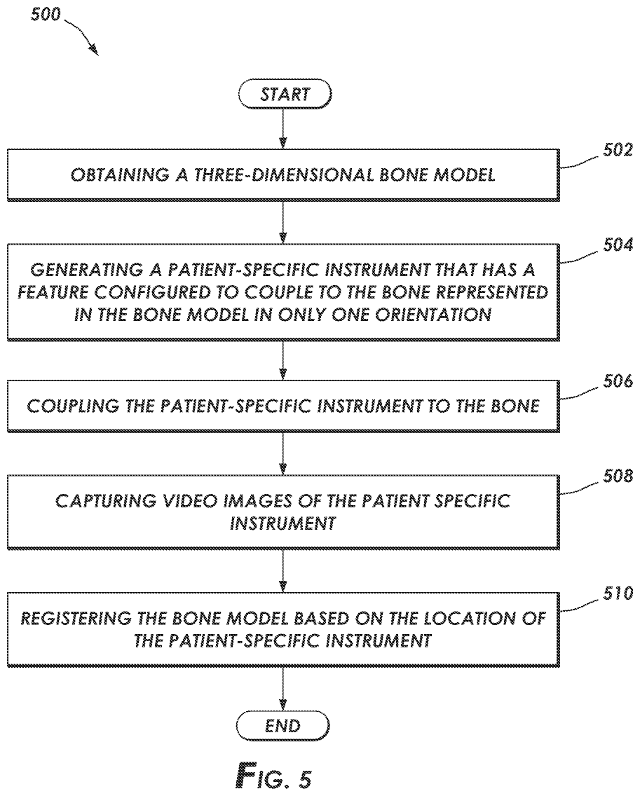

FIG. 5 shows a method 500 in accordance with at least some embodiments. The example method may be implemented in software within one or more computer systems, such as, in part, the surgical controller 118. In particular, the example method 500 starts and comprises obtaining a three-dimensional bone model (block 502). Much like the prior techniques, in the patient-specific instrument registration technique what is obtained is the three-dimensional bone model that may be created by segmenting a plurality of non-invasive images (e.g., MRI) taken preoperatively or intraoperatively.

The next step in the example method 500 is generating a patient-specific instrument that has a feature designed to couple to the bone represented in the bone model in only one orientation (block 504). Generating the patient-specific instrument may first involve selecting a location at which the patient-specific instrument will attach. For example, a device or computer system may analyze the bone model and select the attachment location. In various examples, the attachment location may be a unique location in the sense that, if a patient-specific instrument is made to couple to the unique location, the patient-specific instrument will not couple to the bone at any other location. In the example case of an anterior cruciate ligament repair, the location selected may be at or near the upper or superior portion on the intercondylar notch. If the bone model shows another location with a unique feature, such as a bone spur or other raised or sunken surface anomaly, such a unique location may be selected as the attachment location for the patient-specific instrument.

Moreover, forming the patient-specific instrument may take any suitable form. In one example, a device or computer system may directly print, such as using a 3D printer, the patient-specific instrument. In other cases, the device or computer system may print a model of the attachment location, and the model may then become the mold for creating the patient-specific instrument. For example, the model may be the mold for an injection-molded plastic or casting technique. In some examples, the patient-specific instrument carries one or more fiducials, but as mentioned above, in other cases the patient-specific instrument may itself be tracked and thus carry no fiducials.

The next step in the example method 500 is coupling the patient-specific instrument to the bone, in some cases the patient-specific instrument having the fiducial coupled to an exterior surface (block 506). As previously mentioned, the attachment location for the patient-specific instrument is selected to be unique such that the patient-specific instrument couples to the bone in only one location and in only one orientation. In the example case of an arthroscopic ACL repair, the patient-specific instrument may be inserted arthroscopically. That is, the attachment location may be selected such that a physical size of the patient-specific instrument enables insertion through the ports in the patient's skin. In other case, the patient-specific instrument may be made or constructed of a flexible material that enables the patient-specific instrument to deform for insertion in the surgical site, yet return to the predetermined shape for coupling to the attachment location. However, in open procedures where the skin is cut and pulled away, exposing the bone to the open air, the patient-specific instrument may be a rigid device with fewer size restrictions.

The next step in the example method 500 is capturing video images of the patient-specific instrument (block 508). Here again, the capturing may be performed intraoperatively. In the example case of an arthroscopic anterior cruciate ligament repair, the capturing of video images is by the surgical controller 118 by way of arthroscope 108 and camera 110. However, in open procedures where the skin is cut and pulled away, exposing the bone to the open air, the capturing may be by any suitable camera device, such as one or both cameras of a stereoscopic camera systems, or a portable computing device, such as a tablet or smart-phone device. In such cases, the video images may be provided to the surgical controller 118 in any suitable form.

The next step in the example method 500 is registering the bone model based on the location of the patient-specific instrument (block 510). That is, given that the patient-specific instrument couples to the bone at only one location and in only one orientation, the location and orientation of the patient-specific instrument is directly related to the location and origination of the bone, and thus the coordinate transformations and scaling for the registration may be calculated directly. Thereafter, the example method 500 may end; however, the surgical controller 118 may then use the registered bone model to provide computer-assisted navigation regarding a surgical task or surgical procedure involving the bone.

For example, with the registered bone model the surgical controller 118 may provide guidance regarding a surgical task of a surgical procedure. The specific guidance is dependent upon the surgical procedure being performed and the stage of the surgical procedure. A non-exhaustive list of guidance comprises: changing a drill path entry point; changing a drill path exit point; aligning an aimer along a planned drill path; showing location at which to cut and/or resect the bone; reaming the bone by a certain depth along a certain direction; placing a device (suture, anchor or other) at a certain location; placing a suture at a certain location; placing an anchor at a certain location; showing regions of the bone to touch and/or avoid; and identifying regions and/or landmarks of the anatomy. In yet still other cases, the guidance may include highlighting within a version of the video images displayed on a display device, which can be the arthroscopic display or a see-through display, or by communicating to a virtual reality device or a robotic tool.

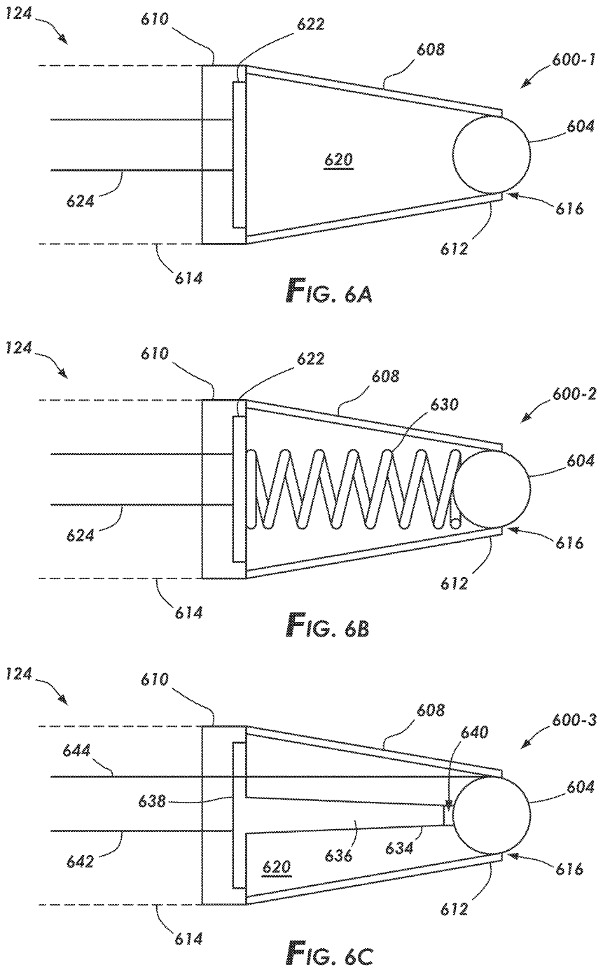

In some examples, systems and methods according to the present disclosure implement a connected or “smart” touch probe and probe tip (referred to collectively as a “probe assembly”) configured to facilitate touch-based registration techniques. For example, a touch probe such as the touch probe 124 may be configured as a smart touch probe or probe assembly as described below in more detail. Various example probe assemblies and probe tips according to the present disclosure are described below with respect to FIGS. 6-9.

As used below in the description of FIGS. 6-13, the term “proximal” refers to a point or direction nearest a handle of the probe (e.g., a direction opposite the probe tip). Conversely, the term “distal” refers to a point or direction nearest the probe tip (e.g., a direction opposite the handle).

As shown in FIGS. 6A, 6B, and 6C, example probe heads or tips 600-1, 600-2, and 600-3 (referred to collectively as probe tips 600) include a roller ball tip or contact ball 604. The contact ball 604 is configured to rotate, with minimal resistance, as the user moves the touch probe (e.g., the touch probe 124 modified to include one of the probe tips 600) against a bone, joint, or other anatomical surface or tissue. As shown, the probe tips 600 include a tapered housing 608 having a base end 610 and a contact end 612. In an example, the base end 610 is configured to couple the probe tip 600 to a shaft or handle 614 of the touch probe 124 (e.g., via complementary threaded interfaces of the base end 610 and the handle 614). In another example, the base end 610 may have a slightly smaller diameter than the handle 614 (or vice versa) and is configured to insert within the handle 614. For example, the base end 610 may be friction-held within the handle 614. In still another example, the probe tip 600 may be integrally formed with the handle 614. Although described as a handle 614, the handle 614 may correspond to a probe shaft or other intermediate mechanical element of the probe 124 coupled to the probe tip 600. For example, the probe tip 600 may be coupled directly to the handle 614, a probe shaft may be coupled between the probe tip 600 and the handle 614, a probe shaft may be configured to function as a handle 614 or vice versa, etc.

The contact end 612 defines an opening 616 and the contact ball 604 is retained within the opening 616. For example, a diameter of the opening 616 is slightly less than a diameter of the contact ball 604 such that an edge or rim of the opening 616 retains the contact ball 604 within the housing 608.

In the example shown in FIG. 6A, the housing 608 encloses a foam element 620 arranged between the contact ball 604 and the base end 610. The foam element 620 may bias the contact ball 604 toward the contact end 612. As the probe tip 600-1 is pressed against an anatomical surface, the contact ball 604 is pushed inward toward and against the foam element 620, which in turn causes the foam element 620 to push against a strain gauge 622 (or, in some examples, a force sensor). The strain gauge 622 may be arranged within the base end 610 (as shown), between the base end 610 and the foam element 620, within the handle 614, etc.

The strain gauge 622 is configured to generate a signal in response to pressure or force exerted by the foam element 620 against the strain gauge 622. In an example, the strain gauge 622 is configured to generate the signal in response to any amount of (i.e., nonzero) force exerted by the foam element 620. In another example, the strain gauge 622 is configured to generate the signal in response to an amount of force exerted by the foam element 620 greater than or equal to a threshold. As shown, the strain gauge 622 is coupled to one or more wires 624 configured to transmit the signal to the surgical system (e.g., the surgical system 100). In other examples, the strain gauge 622 may be coupled to circuitry within the touch probe 124 configured to wirelessly transmit the signal to the surgical system 100.

The surgical system 100 is configured to initiate and perform registration in response to detecting the signal from the strain gauge 622 (e.g., in accordance with the touch-based registration techniques described herein). For example, the surgical system 100 is configured to register a “point” in response to the contact ball 604 being pressed against an anatomical surface. In other examples, the probe tip 600-1 and the contact ball 604 may be used to “paint” the anatomical surface, such as an outer surface of a bone, as described above. In this manner, the probe tip 600-1 can be used to collect points for registering a three-dimensional model without requiring the user to provide additional inputs to the surgical system 100. In other words, detection of the signal from the strain gauge 622 can be performed automatically in response to the contact ball 604 being pressed against the anatomical surface.

In the example shown in FIG. 6B, the housing 608 encloses a spring 630 or other biasing element (i.e., instead of the foam element 620). The spring 630 is arranged between the contact ball 604 and the base end 610. Similar to the example shown in FIG. 6A, the spring 630 biases the contact ball 604 toward the contact end 612. As the probe tip 600-2 is pressed against the anatomical surface, the contact ball 604 is pushed inward toward and against the spring 630, which in turn causes the spring 630 to push against the strain gauge 622.

In the example shown in FIG. 6C, the foam element 620 includes a central opening or channel 634. A contact pin 636 extends from the base end 610 into the channel 634 toward the contact ball 604. For example, the contact pin 636 may include a flange 638 configured to retain the contact pin 636 within the base end 610 against a proximal surface of the foam element 620. As shown, the channel 634 and the contact pin 636 are tapered.

As shown (with the contact ball 604 in an “un-pressed” position), a gap 640 is defined between a distal end of the contact pin 636 and the contact ball 604. As the contact ball 604 is pressed inward against the foam element 620 (i.e., in response to the user pressing the probe tip 601-3 against the anatomical surface), the contact ball 604 engages/contacts the contact pin 636. The contact pin 636 is configured to generate a signal in response to the contact with the contact ball 604.

In an example, the signal is generated in response to a change in resistance caused by contact between the contact pin 636 and the contact ball 604. For example, the contact pin 636 may be comprised of and/or plated in an electrically conductive material, such as copper. Contact between the contact pin 636 and the contact ball 604 completes a circuit between respective wires 642 and 644, causing a signal to be transmitted to the surgical system 100 indicating contact between the contact ball 604 and the anatomical surface.

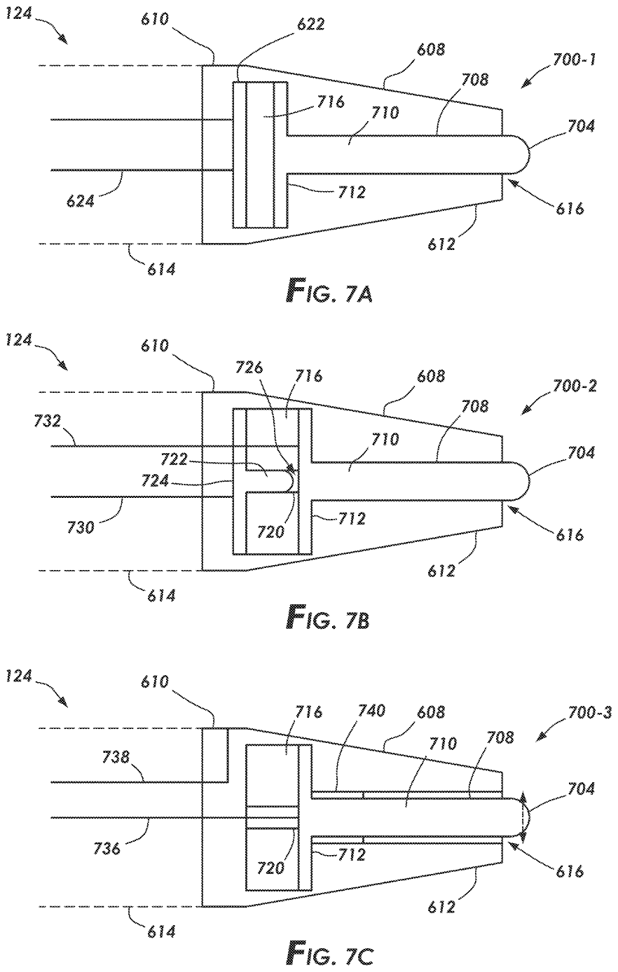

FIGS. 7A, 7B, and 7C show example probe heads or tips 700-1, 700-2, and 700-3 (referred to collectively as probe tips 700). Some reference numbers used for FIGS. 6A, 6B, and 6C are reused to identify similar and/or identical elements. In these examples, the probe tips 700 include a rounded contact or “pen” tip 704. The pen tip 704 is non-rotating. Rather, the pen tip 704 is configured to minimize friction between the pen tip 704 and the anatomical surface. As shown, the probe tips 700 include the tapered housing 608, the base end 610 coupling the probe tip 700 to the handle 614, and the contact end 612. The contact end 612 defines the opening 616 and the pen tip 704 is retained within the opening 616.

In the example shown in FIG. 7A, the pen tip 704 is coupled to or integrally formed with a plunger 708. The plunger 708 includes a rod or shaft 710 that mechanically couples the pen tip 704 to a flange 712. The pen tip 704 is located distally with respect to the probe tip 700-1 while the flange 712 is located proximally with respect to the probe tip 700-1.

The flange 712 is arranged against/adjacent to (i.e., is in contact with) a foam pad 716. In other examples, the foam pad 716 may be replaced by a spring or other biasing element. The plunger 708 extends from the foam pad 716 toward the contact end 612 of the probe tip 700-1. The foam pad 716 is arranged against the strain gauge 622. In other words, as shown, the foam pad 716 is arranged between the flange 712 and the strain gauge 622. As the probe tip 700-1 (i.e., the pen tip 704) is pressed against the anatomical surface, the flange 712 is pushed inward toward and against the foam pad 716, which in turn causes the foam pad 716 to push against the strain gauge 622. The strain gauge 622 is configured to generate a signal in response to pressure or force exerted by the foam pad 716 against the strain gauge 622 as described above in FIGS. 6A and 6B. For example, as shown, the strain gauge 622 is coupled to one or more wires 624 configured to transmit the signal to the surgical system 100. The surgical system 100 is configured to initiate and perform registration in response to detecting the signal from the strain gauge 622.

In the example shown in FIG. 7B, the foam pad 716 includes a central opening or channel 720. A contact pin 722 extends from the base end 610 into the channel 720 toward the plunger 708 (e.g., distally, toward the flange 712). For example, the contact pin 722 may include a flange 724 configured to retain the contact pin 722 within the base end 610 against a proximal surface of the foam pad 716. As shown, a distal end or tip of the contact pin 722 is rounded.

As shown (with the contact ball 604 in an “un-pressed” position), a gap 726 is defined between the distal end of the contact pin 722 and the flange 712. As the pen tip 704 is pressed inward against the foam pad 716 (i.e., in response to the user pressing the probe tip 701-2 against the anatomical surface), the flange 712 engages/contacts the contact pin 722. The contact pin 722 is configured to generate a signal in response to the contact with the flange 712.

In an example, the signal is generated in response to a change in resistance caused by contact between the contact pin 722 and the flange 712. For example, the contact pin 722 may be comprised of and/or plated in an electrically conductive material, such as copper. Contact between the contact pin 722 and the flange 712 completes a circuit between respective wires 730 and 732, causing a signal to be transmitted to the surgical system 100 indicating contact between the pen tip 704 and the anatomical surface.

In the example shown in FIG. 7C, contact between the pen tip 704 and the anatomical surface biases the pen tip 704 in a direction toward the housing 608 of the probe tip 700-3 (e.g., a lateral direction relative to the housing 608, corresponding to a vertical direction in FIG. 7C). In this example, the housing 608 may be comprised of and/or plated in conductive material. Contact between the plunger 708 (e.g., a side of the pen tip 704 or shaft 710) and the housing 608 completes a circuit between respective wires 736 and 738 (e.g., via the plunger 708 and the housing 608), causing a signal to be transmitted to the surgical system 100 indicating contact between the pen tip 704 and the anatomical surface.

In an example (as shown), a diameter of the opening 616 may be slightly larger than a diameter of the shaft 710, enabling the pen tip 704 to move within the opening 616 toward the housing 608. Accordingly, a foam ring or bushing 740 may be arranged around a base of the shaft 710 (e.g., adjacent to the flange 712). The foam bushing 740 provides support for the plunger 708 and may be configured to retain a generally central position of the pen tip 704 within the opening 616.

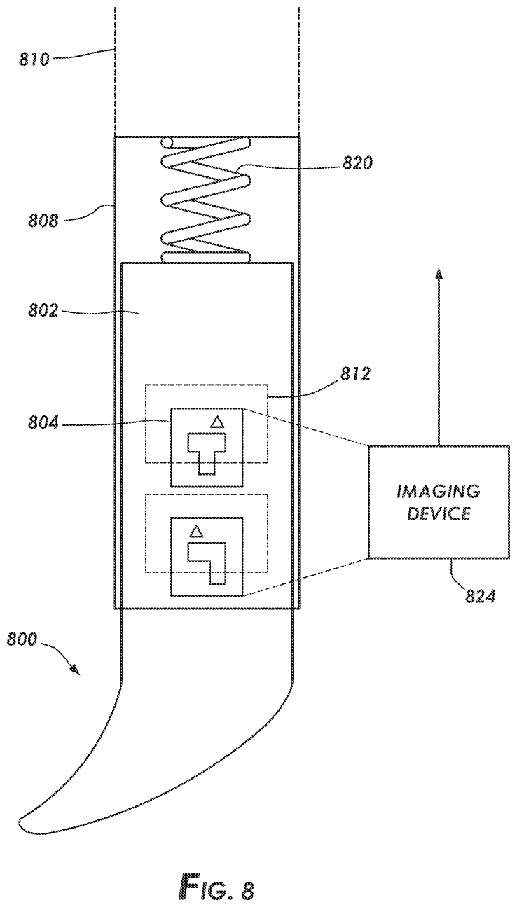

FIG. 8 shows another example probe tip 800 according to the principles of the present disclosure. In this example, a body 802 of the probe tip 800 includes one or more fiducials (e.g., patterns, markings, etc.) 804. The probe tip 800 is partially enclosed and retained within an outer sleeve 808 (which may be coupled to a handle 810 of the touch probe 124). The outer sleeve 808 includes respective windows or openings 812 corresponding to the fiducials 804. In an example, the openings 812 are a same size as or larger than the fiducials 804. Accordingly, the fiducials 804 are viewable/visible through the openings 812.

A spring 820 or other biasing element is arranged at a proximal end of the outer sleeve 808 (e.g., adjacent to the handle 810 or other rigid structure). The spring 820 is arranged against/adjacent to a proximal end of the body 802 of the probe tip 800 and biases the probe tip 800 distally relative to the handle 810, the outer sleeve 808, etc. Accordingly, as shown (with the probe tip 800 in an “un-pressed” position), the spring 820 causes misalignment between the fiducials 804 and the openings 812 and the fiducials 804 are not detectable (e.g., by a viewing or imaging device 824, such as a camera). In other words, when the probe tip 800 is not pressed against the anatomical surface, detection of the fiducials 804 is prevented.

Conversely, as the probe tip 800 is pressed inward against the spring 820 (i.e., in response to the user pressing the probe tip 800 against the anatomical surface), the fiducials 804 become aligned with and visible through the openings 812. The imaging device 824 (or circuitry coupled to the imaging device 824) is configured to generate a signal in response to detection of one or more of the fiducials 804, thereby initiating/performing registration as described herein.

Although shown on one side of the body 802 of the probe tip 800, the fiducials 804 may be arranged on multiple sides of the body 802 and the outer sleeve 808 may include corresponding openings 812. In this manner, views of the fiducials 804 may be obtained from different positions/angles (e.g., with the touch probe 124 in a variety of positions relative to the imaging device 824) to initiate registration. In some examples, two or more of the imaging devices 824 may be used. The imaging device 824 may be in a fixed position, implemented as a moveable or handheld instrument, etc.

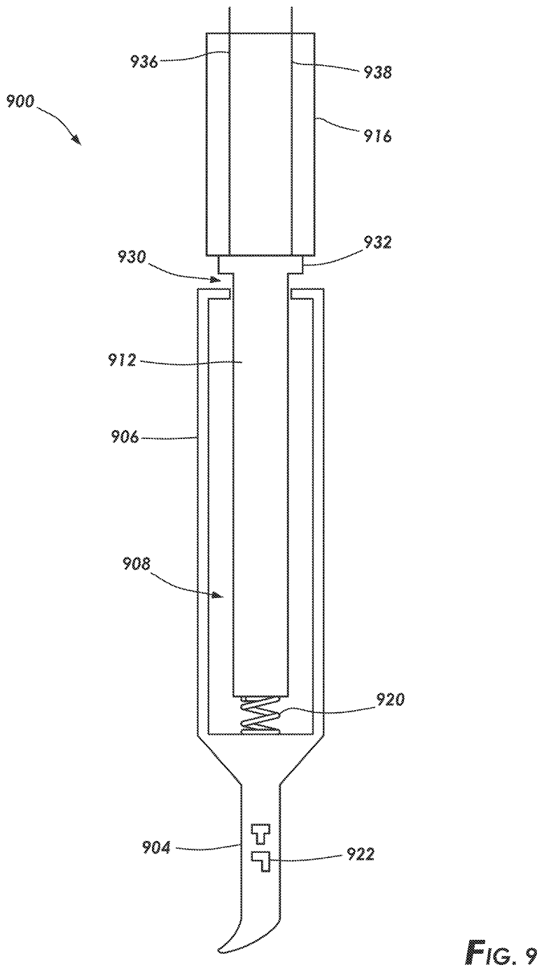

FIG. 9 shows another example probe assembly 900 including a probe tip 904 according to the principles of the present disclosure. In this example, the probe tip 904 is coupled to or integrally formed with an outer sleeve 906. The outer sleeve 906 defines an inner volume 908 configured to receive, enclose, and retain an inner shaft 912 coupled to or integrally formed with a handle 916. A spring 920 or other biasing element is arranged between a distal end of the inner shaft 912 and a proximal end of the probe tip 904. In some examples, the probe tip 904 may include one or more fiducials 922 detectable as described herein.

The spring 920 biases the probe tip 904 in a direction away from the inner shaft 912 and the handle 916 (or, in other words, biases the inner shaft 912 and the handle 916 in a direction away from the probe tip 904). As shown (with the probe tip 904 in an “un-pressed” position), a gap 930 is defined between a proximal end of the outer sleeve 906 and a contact plate or flange 932. As shown, the contact plate 932 is located on a proximal end of the inner shaft 912. In other examples, the contact plate 932 is located on a distal end of the handle 916.

As the probe tip 904 is pressed inward against the spring 920 (i.e., in response to the user pressing the probe tip 900 against the anatomical surface), the outer sleeve 908 (e.g., a contact portion, a contact element arranged on the proximal end of the outer sleeve 908, etc.) engages/contacts the contact plate 932. The contact plate 932 (and/or circuitry coupled to the contact plate 932) is configured to generate a signal in response to the contact with the outer sleeve 908. For example, contact between the contact plate 932 and the outer sleeve 908 completes a circuit between respective wires 936 and 938, causing a signal to be transmitted to the surgical system 100 indicating contact between the probe tip 904 and the anatomical surface. In this and other examples provided herein, the signal may be generated in response to a change in resistance caused by contact between the contact plate 932 and the outer sleeve 908, and the signal may be transmitted via wires, wirelessly, or combinations thereof. Registration is initiated in response to the signal.

In each of the above examples, the systems and methods of the present disclosure facilitate initiation, performance, and completion of the registration procedure.

In some example registration procedures, the anatomical surface is physically contacted/touched with a calibrated instrument to provide, to the surgical system 100, an indication of a location of the anatomical surface (e.g., in given coordinate space) while viewing fiducials arranged on the instrument. In some procedures, points on the anatomical surface may be selectively captured using the instrument as described in various embodiments herein. However, it may not be desirable to capture points during the registration procedure due to various factors (e.g., a requirement to lift the instrument from the anatomical surface, turned a connected tablet or other application on and off, etc.).

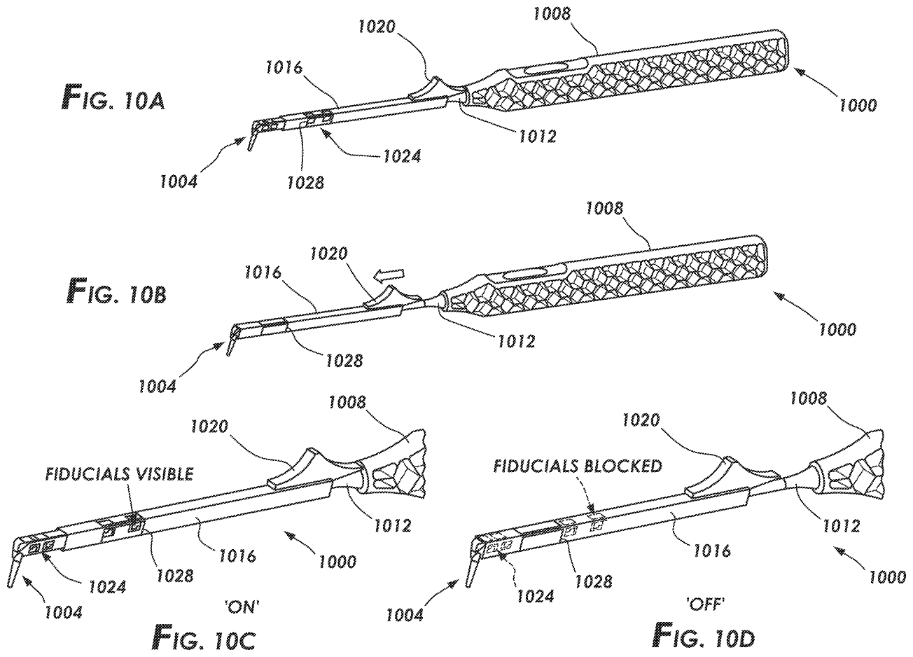

FIGS. 10A, 10B, 10C, and 10D show another example probe assembly 1000 including a probe tip 1004 according to the principles of the present disclosure. In these examples, the probe assembly 1000 is configured to facilitate control of registration of points on the anatomical surface (i.e., control of whether a given point or points are registered while the probe tip 1004 is in contact with the anatomical surface).

The probe assembly 1000 includes a handle 1008 coupled to the probe tip 1004 (e.g., via an inner shaft 1012). An outer sleeve 1016 is arranged on and encloses at least a portion of the probe tip 1004 and the inner shaft 1012. For example, the outer sleeve 1016 has a slidable relationship relative to the probe tip 1004 and the inner shaft 1012. In other words, the outer sleeve 1016 is configured to slide toward and away from the probe tip 1004. In an example, the outer sleeve 1016 includes a mechanical switch (e.g., a thumb switch) 1020 coupled to or integrally formed with the outer sleeve 1016. As shown, the switch 1020 is located at a proximal end of the outer sleeve 1016. In some examples, the outer sleeve 1016 may be biased (e.g., using a spring) in a first direction toward or away from the probe tip 1004 and force is required to slide the outer sleeve 1016 in a second direction opposite the first direction. In other examples, the outer sleeve 1016 may be friction-held in a position relative to the probe tip 1004.

The probe tip 1004 (e.g., a proximal portion of the probe tip 1004 retained within the outer sleeve 1016 includes one or more fiducials 1024. The fiducials 1024 are selectively viewable/visible (e.g., using an imaging device such as described herein) or obscured/blocked by the outer sleeve 1016. For example, the fiducials 1024 may be selectively exposed beyond a distal end of the outer sleeve 1016 and/or through one or more windows or openings 1028 defined in the outer sleeve 1016. As shown in FIGS. 10A and 10C, the fiducials 1024 are visible (i.e., not blocked by the outer sleeve 1016). Conversely, as shown in FIGS. 10B and 10D, the fiducials 1024 are blocked/obscured by the outer sleeve 1016). Accordingly, in this manner, the fiducials 1024 can be selectively visible or blocked based on the position of the outer sleeve 1016.

In an example, in the “blocked” (or “off”) position as shown in FIGS. 10B and 10D, the fiducials 1024 are not detectable by the surgical system 100 (i.e., using an associated imaging device). Accordingly, even though the probe tip 1004 may be in contact with the anatomical surface, points are not registered by the surgical system 100. Conversely, in the “open” (or “on”) position as shown in FIGS. 10A and 10C, the fiducials 1024 are detectable by the surgical system and points can by registered using various techniques as described herein.

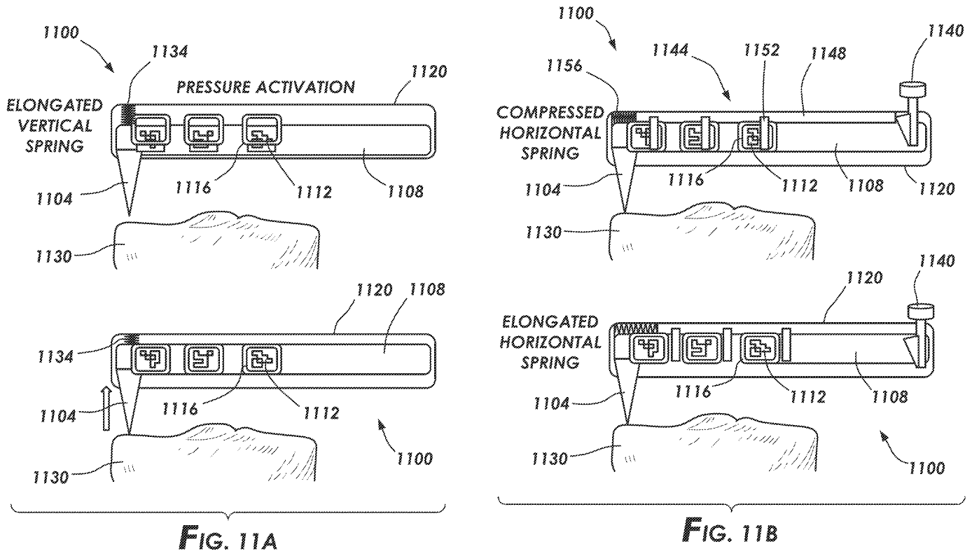

FIGS. 11A and 11B show another example probe assembly 1100 including a probe tip 1104 according to the principles of the present disclosure. Similar to the example shown in FIGS. 10A, 10B, 10C, and 10D, the probe tip 1104 is couple to an inner shaft 1108 having one or more fiducials 1112. The fiducials 1112 are selectively viewable via respective openings 1116 defined in an outer sleeve 1120. Various mechanisms may be used to selectively view/expose or block the fiducials 1112.

As shown in FIG. 11A, the fiducials 1112 may be in a “normally blocked” state when the probe tip 1104 is not in contact with or pressed against an anatomical surface (e.g., a surface of a bone 1130). For example, the probe assembly 1100 may include a spring 1134 or other biasing element arranged between the inner shaft 1108 and the outer sleeve 1120. The spring 1134 biases the outer sleeve 1120 away from the inner shaft 1108 (e.g., in a first direction away from the probe tip 1104), causing misalignment between the fiducials 1112 and the openings 1116. Conversely, when the probe tip 1104 is pressed against the anatomical surface, the spring 1134 is compressed and the outer sleeve 1120 is moved in a second direction toward the probe tip 1104. In this manner, the fiducials 1112 become viewable and detectable through the openings 1116.

Conversely, as shown in FIG. 11B, the probe assembly 1100 includes a button 1140 and blocking assembly 1144. The blocking assembly 1144 includes a slidable shaft 1148 arranged within the outer sleeve 1120 (e.g., between the inner shaft 1108 and the outer sleeve 1120). Respective bars or posts 1152 extend (e.g., perpendicularly) from the slidable shaft 1148 to cover at least a portion of the fiducials 1112. In a “blocked” or “off” state or position, the button 1140 (e.g., wedge element of the button) biases the blocking assembly 1144 toward a distal end of the probe assembly 1100 (i.e., an end of the probe assembly 1100 including the probe tip 1104) and away from the button 1140. In the blocked position, a spring 1156 arranged between a distal end of the blocking assembly 1144 and a distal end of the outer sleeve 1120 is compressed.

The button 1140 can be selectively actuated or pressed, releasing a proximal end of the blocking assembly 1144 from the wedge element of the button 1140 and allowing the spring 1156 to bias the blocking assembly 1144 away from the distal end of the probe assembly 1100 and toward the button 1140. In other words, the button 1140 can be pressed to transition the probe assembly 1100 to an “open” or “on” state/position. In the open position, the posts 1152 do not cover/extend over the fiducials 1112, allowing the fiducials 1112 to be viewed/detected.

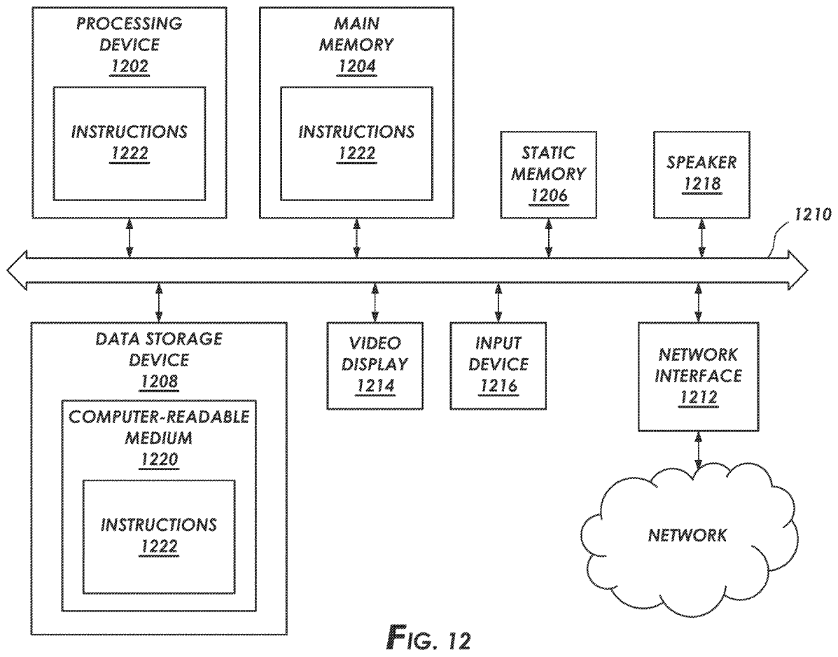

FIG. 12 shows an example computer system 1200. In one example, computer system 1200 may correspond to the surgical controller 118, device that creates the patient-specific instrument, a tablet device within the surgical room, or any other system that implements any or all the various methods discussed in this specification. The computer system 1200 may be connected (e.g., networked) to other computer systems in a local-area network (LAN), an intranet, and/or an extranet (e.g., device cart 102 network), or at certain times the Internet (e.g., when not in use in a surgical procedure). The computer system 1200 may be a server, a personal computer (PC), a tablet computer or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 1200 includes a processing device 1202, a main memory 1204 (e.g., read-only memory (ROM), flash memory, dynamic random access memory (DRAM) such as synchronous DRAM (SDRAM)), a static memory 1206 (e.g., flash memory, static random access memory (SRAM)), and a data storage device 1208, which communicate with each other via a bus 1210.

Processing device 1202 represents one or more general-purpose processing devices such as a microprocessor, central processing unit, or the like. More particularly, the processing device 1202 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1202 may also be one or more special-purpose processing devices such as an application specific integrated circuit (ASIC), a field programmable gate array (FPGA), a digital signal processor (DSP), network processor, or the like. The processing device 1202 is configured to execute instructions for performing any of the operations and steps discussed herein. Once programmed with specific instructions, the processing device 1202, and thus the entire computer system 1200, becomes a special-purpose device, such as the surgical controller 118.

The computer system 1200 may further include a network interface device 1212 for communicating with any suitable network (e.g., the device cart 102 network). The computer system 1200 also may include a video display 1214 (e.g., display device 414), one or more input devices 1216 (e.g., a microphone, a keyboard, and/or a mouse), and one or more speakers 1218. In one illustrative example, the video display 1214 and the input device(s) 1216 may be combined into a single component or device (e.g., an LCD touch screen).

The data storage device 1208 may include a computer-readable storage medium 1220 on which the instructions 1222 (e.g., implementing any methods and any functions performed by any device and/or component depicted described herein) embodying any one or more of the methodologies or functions described herein is stored. The instructions 1222 may also reside, completely or at least partially, within the main memory 1204 and/or within the processing device 1202 during execution thereof by the computer system 1200. As such, the main memory 1204 and the processing device 1202 also constitute computer-readable media. In certain cases, the instructions 1222 may further be transmitted or received over a network via the network interface device 1212.

While the computer-readable storage medium 1220 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The computer system 1200 or one or more computing or processing devices may be configured to perform functions of the registration procedure described herein, including functions related to communication and/or control of any of the probes described in FIGS. 6-11, and/or functions of the methods described herein.

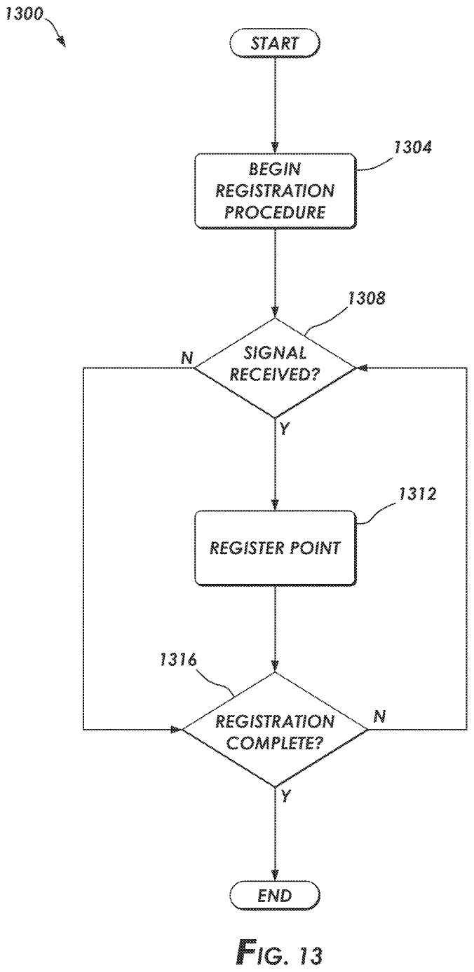

FIG. 13 illustrates steps of an example method 1300 for performing a touch-based registration procedure (e.g., a touch-based registration procedure performed using any of the probes described herein, such as the probes described in FIGS. 6-11). The method 1300 can be performed in conjunction with any of the other methods described herein.

The registration procedure begins at 1304. For example, the registration procedure may begin in response to a command from a user (e.g., at the surgical system 100 or an associated computer system or computing device, application, etc.). In some examples, the registration procedure may be being in response to receiving a signal from a touch probe as described herein.

At 1308, the method 1300 includes determining whether a signal indicating contact with an anatomical surface is received from a touch probe and/or a device or circuitry associated with and in communication with the touch probe. For example, the signal may correspond to a signal generated in response to a contact ball or probe tip being pressed inward (as in FIGS. 6A, 6B, 6C, and 9), in response to a pen tip being pressed inward or against a housing of a probe tip (as in FIGS. 7A, 7B, and 7C), in response to a probe tip being pressed inward and exposing one or more fiducials for viewing by an imaging device (as in FIGS. 8 and 11A), in response to one or more fiducials being exposed via a mechanical switch or button (as in FIGS. 10A, 10B, 10C, 10D, and 11B), etc. If true, the method 1300 proceeds to 1312. If false, the method 1300 proceeds to 1316.

At 1312, the method 1300 includes registering a point based on a position of the touch probe. For example, registering the point includes registering a point of a three-dimensional bone model in accordance with any of the techniques described herein.

At 1316, the method 1300 includes determining whether the registration procedure is complete. If true, the method 1300 ends. If false, the method 1300 continues to 1308.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In some implementations, a controller is part of a system, which may be part of the above-described examples. Such systems can comprise semiconductor processing equipment, including a processing tool or tools, chamber or chambers, a platform or platforms for processing, and/or specific processing components (a wafer pedestal, a gas flow system, etc.). These systems may be integrated with electronics for controlling their operation before, during, and after processing of a semiconductor wafer or substrate. The electronics may be referred to as the “controller,” which may control various components or subparts of the system or systems. The controller, depending on the processing requirements and/or the type of system, may be programmed to control any of the processes disclosed herein, including the delivery of processing gases, temperature settings (e.g., heating and/or cooling), pressure settings, vacuum settings, power settings, radio frequency (RF) generator settings, RF matching circuit settings, frequency settings, flow rate settings, fluid delivery settings, positional and operation settings, wafer transfers into and out of a tool and other transfer tools and/or load locks connected to or interfaced with a specific system.

Broadly speaking, the controller may be defined as electronics having various integrated circuits, logic, memory, and/or software that receive instructions, issue instructions, control operation, enable cleaning operations, enable endpoint measurements, and the like. The integrated circuits may include chips in the form of firmware that store program instructions, digital signal processors (DSPs), chips defined as application specific integrated circuits (ASICs), and/or one or more microprocessors, or microcontrollers that execute program instructions (e.g., software). Program instructions may be instructions communicated to the controller in the form of various individual settings (or program files), defining operational parameters for carrying out a particular process on or for a semiconductor wafer or to a system. The operational parameters may, in some embodiments, be part of a recipe defined by process engineers to accomplish one or more processing steps during the fabrication of one or more layers, materials, metals, oxides, silicon, silicon dioxide, surfaces, circuits, and/or dies of a wafer.

The controller, in some implementations, may be a part of or coupled to a computer that is integrated with the system, coupled to the system, otherwise networked to the system, or a combination thereof. For example, the controller may be in the “cloud” or all or a part of a fab host computer system, which can allow for remote access of the wafer processing. The computer may enable remote access to the system to monitor current progress of fabrication operations, examine a history of past fabrication operations, examine trends or performance metrics from a plurality of fabrication operations, to change parameters of current processing, to set processing steps to follow a current processing, or to start a new process. In some examples, a remote computer (e.g. a server) can provide process recipes to a system over a network, which may include a local network or the Internet. The remote computer may include a user interface that enables entry or programming of parameters and/or settings, which are then communicated to the system from the remote computer. In some examples, the controller receives instructions in the form of data, which specify parameters for each of the processing steps to be performed during one or more operations. It should be understood that the parameters may be specific to the type of process to be performed and the type of tool that the controller is configured to interface with or control. Thus as described above, the controller may be distributed, such as by comprising one or more discrete controllers that are networked together and working towards a common purpose, such as the processes and controls described herein. An example of a distributed controller for such purposes would be one or more integrated circuits on a chamber in communication with one or more integrated circuits located remotely (such as at the platform level or as part of a remote computer) that combine to control a process on the chamber.

Without limitation, example systems may include a plasma etch chamber or module, a deposition chamber or module, a spin-rinse chamber or module, a metal plating chamber or module, a clean chamber or module, a bevel edge etch chamber or module, a physical vapor deposition (PVD) chamber or module, a chemical vapor deposition (CVD) chamber or module, an atomic layer deposition (ALD) chamber or module, an atomic layer etch (ALE) chamber or module, an ion implantation chamber or module, a track chamber or module, and any other semiconductor processing systems that may be associated or used in the fabrication and/or manufacturing of semiconductor wafers.

As noted above, depending on the process step or steps to be performed by the tool, the controller might communicate with one or more of other tool circuits or modules, other tool components, cluster tools, other tool interfaces, adjacent tools, neighboring tools, tools located throughout a factory, a main computer, another controller, or tools used in material transport that bring containers of wafers to and from tool locations and/or load ports in a semiconductor manufacturing factory.

1. A probe for registering an anatomical surface for a registration procedure, the probe comprising:
a probe shaft;
a probe tip having a base end coupled to a distal end of the probe shaft;
a contact ball disposed and configured to rotate within a contact end of the probe tip; and
a biasing element disposed within the probe tip, wherein the biasing element is configured to bias the contact ball toward the contact end of the probe tip, and wherein the contact ball is configured to, in response to external force against the contact ball, push against the biasing element,
wherein the probe is configured to generate a signal in response to the contact ball pushing against the biasing element.

2. The probe of claim 1, wherein the biasing element includes at least one of a foam element and a spring arranged between the contact ball and a strain gauge.

3. The probe of claim 2, wherein the strain gauge is configured to cause the signal to be generated in response to the external force having a nonzero value.

4. The probe of claim 2, wherein the strain gauge is configured to cause the signal to be generated in response to the external force exceeding a threshold.

5. The probe of claim 1, wherein the biasing element is a foam element, wherein the foam element includes a channel, and wherein the probe further comprises a contact pin extending from the base end toward the contact end through the channel.

6. The probe of claim 5, wherein the contact ball is configured to be pressed against the foam element to contact the contact pin, and wherein the probe is configured to generate the signal in response to contact between the contact ball and the contact pin.

7. A probe for registering an anatomical surface for a registration procedure, the probe comprising:
a probe shaft;
a probe tip having (i) a contact end and (ii) a base end coupled to a distal end of the probe shaft;
a plunger disposed within the probe tip, wherein the plunger includes a flange at a proximal end of the plunger and a contact tip at a distal end of the plunger; and
a biasing element disposed within the probe tip, wherein the biasing element is configured to bias the plunger toward the contact end of the probe tip, and wherein the plunger is configured to, in response to external force against the contact tip, push against the biasing element,
wherein the probe is configured to generate a signal in response to the plunger pushing against the biasing element.

8. The probe of claim 7, wherein the biasing element includes a foam element arranged between the plunger and a strain gauge.

9. The probe of claim 8, wherein the strain gauge is configured to cause the signal to be generated in response to the external force having a nonzero value.

10. The probe of claim 8, wherein the strain gauge is configured to cause the signal to be generated in response to the external force exceeding a threshold.

11. The probe of claim 7, wherein the biasing element is a foam element arranged between the plunger and a contact pin, and wherein the probe is configured to generate the signal in response to the plunger contacting the contact pin.

12. The probe of claim 7, wherein the probe is configured to generate the signal in response to contact between the plunger and a housing of the probe tip.

13-35. (canceled)

36. A probe for registering an anatomical surface for a registration procedure, the probe comprising:
a probe shaft;
a probe tip having (i) a contact end and (ii) a base end coupled to a distal end of the probe shaft;
a contact element disposed within the contact end of the probe tip; and
a biasing element disposed within the probe tip, wherein the biasing element is configured to bias the contact element toward the contact end of the probe tip, and wherein the contact element is configured to, in response to external force against the contact element, push against the biasing element,
wherein the probe is configured to generate a signal in response to the contact element pushing against the biasing element.

37. The probe of claim 36, wherein the contact element is a contact ball disposed and configured to rotate within the contact end of the probe tip.

38. The probe of claim 36, wherein the biasing element includes at least one of a foam element and a spring arranged between the contact ball and a strain gauge, and wherein at least one of:
the strain gauge is configured to cause the signal to be generated in response to the external force having a nonzero value; and
the strain gauge is configured to cause the signal to be generated in response to the external force exceeding a threshold.

39. The probe of claim 36, wherein the biasing element is a foam element, wherein the foam element includes a channel, and wherein the probe further comprises a contact pin extending from the base end toward the contact end through the channel.

40. The probe of claim 39, wherein the contact ball is configured to be pressed against the foam element to contact the contact pin, and wherein the probe is configured to generate the signal in response to contact between the contact ball and the contact pin.

41. The probe of claim 36, wherein the contact element is a plunger disposed within the probe tip, wherein the plunger includes a flange at a proximal end of the plunger and a contact tip at a distal end of the plunger.

42. The probe of claim 41, wherein the biasing element includes a foam element arranged between the plunger and a strain gauge, and wherein at least one of:
the strain gauge is configured to cause the signal to be generated in response to the external force having a nonzero value; and
the strain gauge is configured to cause the signal to be generated in response to the external force exceeding a threshold.

43. The probe of claim 41, wherein at least one of:
the biasing element is a foam element arranged between the plunger and a contact pin, and wherein the probe is configured to generate the signal in response to the plunger contacting the contact pin; and
the probe is configured to generate the signal in response to contact between the plunger and a housing of the probe tip.
